(19) United States
(12) Patent Application Publication
(10) Pub. No.: US 2009/0182399 A1
(43) Pub. Date: Jul. 16, 2009

(54) AURICULAR THERMOREGULATION SYSTEM FOR APPETITE SUPPRESSION

(76) Inventor: Diana Sylvestre, Oakland, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/351,662

(22) Filed: Jan. 9, 2009

Related U.S. Application Data
(60) Provisional application No. 61/020,627, filed on Jan. 11, 2008.

(51) Int. Cl. A61F 7/12

(52) U.S. Cl. 607/99; 607/113

(57) ABSTRACT

Disclosed herein is a system to stimulate at least a part of the vestibular system of a patient to induce a sensation of anorexia to promote weight loss. The system can include a thermal element, a power source, a conduit, and a control unit configured to regulate the temperature of the thermal element, the duration in which the thermal element is applied, the duration in between thermal stimuli, and/or the number of stimulus cycles to promote a sensation of anorexia in a patient while avoiding or minimizing undesirable side effects such as nausea, vomiting, nystagmus, or vertigo. Methods of using the system are also disclosed.

The present application claims priority under 35 U.S.C. §119(e) to U.S. Provisional Patent Application Ser. No. 61/020,627, entitled “Auricular Thermoregulation System for Appetite Suppression,” filed Jan. 11, 2008. The entire disclosure of the priority application is hereby expressly incorporated by reference in its entirety.

### BACKGROUND OF THE INVENTION

1. Field of the Invention

The present application pertains to methods and apparatuses for stimulating the vestibular system. More particularly, the application pertains to caloric stimulation of the vestibular system to induce a state of anorexia, or appetite suppression, as a technique for weight loss and management.

2. Description of the Related Art

It is well known that achieving and maintaining a desired weight is highly sought out by a significant proportion of the population. To assist people in managing their weight, a variety of approaches have been utilized for both medical and personal reasons, including diet, medication and surgical procedures.

Numerous dietary regimens have been developed, including multi-step programs that require patients to follow daily or weekly routines. Such dietary regimens may be costly and result in variable patient compliance. Many of these diets are also simply ineffective.

Pharmacologic therapy has also developed to assist the general population with weight control. Many of these medications are used to suppress appetite. The use of drugs to suppress appetite often requires ingestion of a pharmaceutical agent once, twice, three times, four times, or more that may be highly addictive or have other adverse effects. Because these drugs are intended to alter one of the fundamental processes of the human body, they are typically prescribed only in cases of morbid obesity, where weight loss can be potentially life-saving, although some drugs are now available over the counter. In certain sub-groups of the population, namely diabetic patients, a meta-analysis of randomized controlled trials by the international Cochrane Collaboration concluded that fluoxetine, orlistat, and sibutramine can achieve statistically significant weight loss over 12 to 57 weeks. The magnitude of weight loss is modest, however, and the long-term health benefits remain unclear.

Drugs for weight control can operate through one or more of the following mechanisms: (1) suppression of appetite. Epilepsy medications and catecholamines and their derivatives (such as amphetamine-based drugs) are the main tools used for this; (2) increase of the body's metabolism; and (3) interference with the body's ability to absorb specific nutrients in food. For example, Orlistat (also known as Xenical and Allī) blocks fat breakdown and thereby prevents fat absorption. The over the counter (OTC) fiber supplements glucomannan and guar gum have been used for the purpose of inhibiting digestion and lowering caloric absorption. Anorectics (also known as anorexigenics) are primarily intended to suppress the appetite, but most of the drugs in this class also act as stimulants (e.g., dexedrine), and patients have abused drugs “off label” to suppress appetite (e.g. digoxin).

Drugs for weight control can have serious side effects. For example, malabsorptive agents such as orlistat may cause frequent, spotting bowel movements, oily stools, stomach pain, flatulence, and gastrointestinal upset. A similar medication, designed for patients with Type 2 diabetes, is Acarbose which partially blocks absorption of carbohydrates in the small intestine, and produces similar side effects including stomach pain, and flatulence. Anorectic agents, such as sibutramine may increase blood pressure and may cause dry mouth, constipation, headache, and insomnia. Byetta (Exenatide) is a long-acting analogue of the hormone GLP-1, which the intestines secrete in response to the presence of food. Drawbacks of Byetta include that it must be injected twice daily, and that it causes severe nausea in some patients, especially when therapy is initiated. A similar agent, Symlin (pramlintide) is a synthetic analogue of the hormone Amylin, which in normal people is secreted by the pancreas in response to eating. Symlin also requires injection, which patients may perceive as undesirable. Other weight loss drugs have also been associated with medical complications, such as fatal pulmonary hypertension and heart valve damage due to Redux and Fen-phen, and hemorrhagic stroke due to phenylpropanolamine. Many of these substances are related to amphetamine. In general, stimulants carry a risk of high blood pressure, faster heart rate, palpitations, closed-angle glaucoma, drug addiction, restlessness, agitation, and insomnia.

There are also many surgical approaches to weight control. For example, one surgical procedure involves reducing the stomach volume in a patient by implanting one or more bulking prostheses into a patient's gastrointestinal tract to provoke a sense of satiety. Also, bariatric surgery is a treatment for morbid obesity that involves alteration of a patient's digestive tract to encourage weight loss and to help maintain normal weight. Known bariatric surgery procedures include jejuno-ileal bypass, jejuno-colic shunt, biliopancreatic diversion, gastric bypass, Roux-en-Y gastric bypass, gastroplasty, gastric banding, vertical banded gastroplasty, and silastic ring gastroplasty. Such surgical procedures are invasive and have a high incidence of morbidity as well as mortality. Complications such as dumping syndrome, malabsorption, infection, adhesions, anastomotic failure, and the like are common.

### SUMMARY OF THE INVENTION

The present application pertains to methods and apparatuses for using mild, controlled caloric stimulation of the vestibular apparatus to induce a state of anorexia in a patient. Anorexia is a loss or reduction in appetite for food, and may benefit individuals seeking to manage their weight. By inducing anorexia in an individual, the individual's urge to consume food will be lessened. By reducing the individual's urge to consume food, it is possible for an individual to control his or her weight, for example, by consuming less calories. This application provides methods and apparatuses to achieve a controlled state of anorexia by using a heating device to stimulate the vestibular apparatus.

In one aspect, methods are provided for stimulating the vestibular system. In some embodiments, a heating device is placed proximate to the ear canal of a patient where it generates heat. The heating device is connected to a power supply source. By generating heat, a thermal differential is created such that heat is transferred across the middle ear, stimulating the vestibular apparatus. In one embodiment, the vestibular apparatus is stimulated so as to induce a state of anorexia in the patient. The methods can be used to manage weight in a patient.

In another aspect, methods are provided for stimulating the vestibular system in which a resistor is placed proximate to an ear of a patient. The resistor is electrically connected to a power supply and generates heat that is transmitted into the ear and stimulates the vestibular system. In some embodiments, the methods are used for weight loss and management.

In some embodiments, a control unit is used to modulate the amount of heat being emitted from the resistor. The control unit can also be used to control the amount of heat released in different time periods, and therefore produce an intermittent thermal differential. This can prevent possible desensitization and loss of response due to over stimulation of the vestibular apparatus.

In some embodiments, methods of suppressing the appetite of a person are provided comprising first identifying a person in need of weight management. A heating device electrically connected to a power supply is placed proximate to an inner ear of the person. The power supply is turned on to generate heat and the heat is applied to the inner ear. Using a control unit, the heat is controlled from the heating device such that the person's appetite is suppressed.

In some embodiments, methods for inducing anorexia of a patient are provided comprising inserting a resistor electrically connected to a power supply proximate to an ear canal of the patient, turning on the power supply to generate heat in the resistor, applying the heat from the resistor to heat the ear canal and controlling the heat from the resistor using a control unit to induce a sensation of anorexia in the patient.

In some embodiments, an apparatus for managing weight in a person is provided comprising a heating element configured to be placed proximate to an ear of the person and a control unit configured to control the amount and duration of heat being generated in the heating element so as to induce a sensation of anorexia in the person when the heating element is placed proximate to the ear of the person.

### BRIEF DESCRIPTION OF THE DRAWINGS

These and other features, aspects and advantages of the various devices, systems and methods presented herein are described with reference to drawings of certain embodiments, which are intended to illustrate, but not to limit, such devices, systems, and methods. It is to be understood that the attached drawings are for the purpose of illustrating concepts of the embodiments discussed herein and may not be to scale.

FIG. 1 is an illustration of the vestibular apparatus found in an inner ear.

FIG. 2 illustrates a circuit design of one embodiment of the present invention.

FIG. 3 illustrates a circuit design of one embodiment of the present invention, including a control unit.

FIG. 4 illustrates one embodiment of the present invention as shown in the ear of a patient.

FIG. 5 shows a schematic diagram of steps a physician may take when using the present invention to treat a patient.

FIG. 6 illustrates a schematic diagram of a vestibular thermal stimulation system in use in the ear of the patient, according to one embodiment of the invention.

FIG. 7 shows a bar graph illustrating the average weekly weight-loss of subjects during an eight-week study involving the use of one embodiment of a thermoregulation device as described herein.

### DETAILED DESCRIPTION OF THE PREFERRED EMBODIMENTS

Methods and apparatuses are provided for using caloric stimulation of the vestibular system to induce a state of anorexia (loss of appetite). The methods and apparatuses can be used as a technique for weight loss and management. Undesired effects such as, for example, nausea, vomiting, and vertigo caused by excessive vestibular stimulation are preferably avoided or at least minimized.

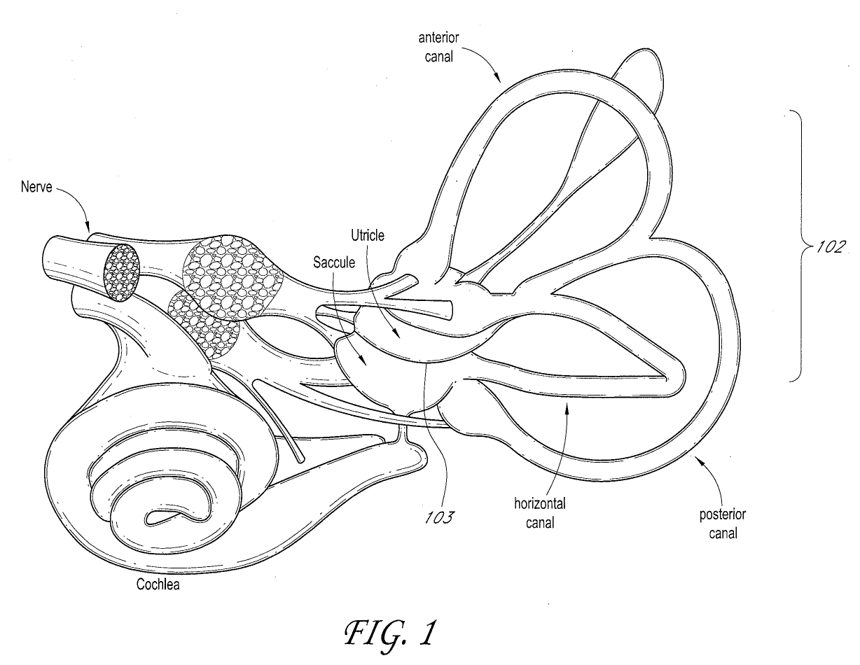

The vestibular system is the sensory system that provides the dominant input about movement and equilibrioception (sense of balance). FIG. 1 shows the vestibular apparatus, located in the labyrinth, or inner ear, which includes otoliths 103 and three semicircular canals (anterior, posterior and horizontal) 102. Otoliths 103 are found in the utricle and saccule of the inner ear, and are responsible for sensing linear translations. The three semicircular canals 102, each of which is located in a different plane in space, are responsible for detecting rotational movements.

Balanced neural input from the vestibular apparatus of the inner ears is primarily responsible for the normal perceptions of movement and orientation in space. Abnormal stimulation of the vestibular apparatus, either by rapid rotation of the head or by differential caloric stimulation, which produces a convection current of endolymph, can initiate a continuum of physical perceptions beginning with anorexia, followed by nausea, vomiting and vertigo.

Caloric stimulation can be used to test for an intact vestibular system. Typically, in this technique, a single ear canal is irrigated with cool or warm water to elicit a clinically apparent response, such as nystagmus. Because of inter-individual variability in the size of the external ear canal and the efficiency of thermal energy transfer across the middle ear, the optimal temperature for eliciting a clinically apparent response may vary. Typically, altering the temperature of the ear canal by no more than about 7, 6, 5, 4, 3, 2, 1, or less degrees Celsius above or below body temperature is adequate.

Without being restricted to a particular theory, the Applicant has discovered that the symptoms of different vestibular stimulation occur on a continuum, and that a mild, controlled caloric stimulation in the form of heating or cooling (e.g. by depositing a heated or chilled liquid) a single ear canal may be used to induce a state of anorexia without eliciting more undesired side effects such as nausea, vomiting, vertigo, and nystagmus. These undesired side effects can occur with more prolonged stimulation, such as a greater temperature differential, a longer exposure to that temperature differential, or more cycles of exposure to that temperature differential.

Mild, controlled thermal stimulation on, in or around the inner ear canal can be used to stimulate the vestibular apparatus. This stimulation preferably elicits anorexia and thus may be used to influence food consumption and control body weight, for example, to assist with desired weight loss. In addition to thermal heating, controlled cooling of the vestibular system, e.g., via a cooling circuit with a solid, liquid, or gas media, such as water, an alcohol, dry ice, or liquid nitrogen may be used to induce anorexia via stimulation of the vestibular apparatus, with parameters that can be adjusted from those described herein for heating.

In one embodiment, anorexia may be induced in a patient by using a device to alter the temperature of the inner ear and stimulate the vestibular apparatus. In some embodiments, the vestibular apparatus may be stimulated by modulating the temperature of the inner ear by heating by no more than about 40, 30, 25, 20, 15, 10, 9, 8, 7, 6, 5, 4.5, 4, 3.5, 3, 2.5, 2, 1.5, 1 degree Celsius, or less. Depending on the type of temperature modulating device used, its proximity to the ear of a patient, and its ability to generate focused heat in a specific location, the time required to modulate the temperature of the inner ear and thus stimulate the vestibular apparatus may vary between mere seconds (e.g., less than 30 seconds) to up to 24 hours, or more. In some embodiments, the vestibular apparatus may be stimulated by modulating the temperature of the inner ear by cooling by no more than about 40, 30, 25, 20 15, 10, 9, 8, 7, 6, 5, 4.5,4, 3.5, 3, 2.5, 2, 1.5, 1 degree Celsius, or less. In some embodiments, the device may be used immediately upon the onset of hunger. In other embodiments, the device may be used consistently at a specific time or multiple times daily, such as before bedtime, or before or after meals or snacks, which will vary depending on an individual's daily schedule of food consumption. For example, in some embodiments, the device may be used at least about, or no more than about 3 hours, 2 hours, 1 hour, 45 minutes, 30 minutes, 15 minutes, 5 minutes, 3 minutes or 1 minute before an individual's routine time of food consumption.

By using a device to alter the temperature of the inner ear and stimulate the vestibular apparatus, a patient's appetite may be suppressed. In some embodiments, appetite suppression results in a patient eating at least about 5%, 10%, 15%, 30%, 35%, 30%, 35%, 50%, or less food compared to when not using the device. In some embodiments, the appetite suppression is transient, such that the patient may experience a normal or greater appetite after a certain period of time. In embodiments in which the appetite suppression is transient, the appetite suppression may last for no more than about 1, 2, 3, 4, 5, 6, 7, 8, 9, 10, 20, 30, 40, 50 minutes, or more while in some embodiments, the appetite suppression may last for no more than about 1, 2, 3, 4, 5, 6, 7, 8, 9, or 10 hours, or more. Transient appetite suppression may be advantageous in that it can be sufficient to allow a spontaneous craving or desire to eat and/or drink, e.g., ingesting an unhealthy snack, to abate such that the patient concentrates on another activity and may not be hungry again until the next mealtime, thus potentially reducing overall daily caloric intake.

The methods disclosed herein may be practiced under the direct supervision of a physician, although in some circumstances, an individual user may be able to use the auricular thermoregulation device described herein without the constant monitoring presence of a physician, such as in the comfort of one's home. For example, upon the onset of hunger, an individual can use the device to provide detectable heat to the individual's own ear so as to suppress his or her own appetite. In some embodiments, safety features may be built into the device to avoid damage to the patient, for example, an automatic shut off feature for the device to avoid overstimulation or damage to the vestibular system or ear, for example, designed to turn the device off if the temperature reaches a certain threshold temperature. The terms “individual,” “user” and “patient” may be used interchangeably to describe one that can be treated by the systems and methods disclosed herein.

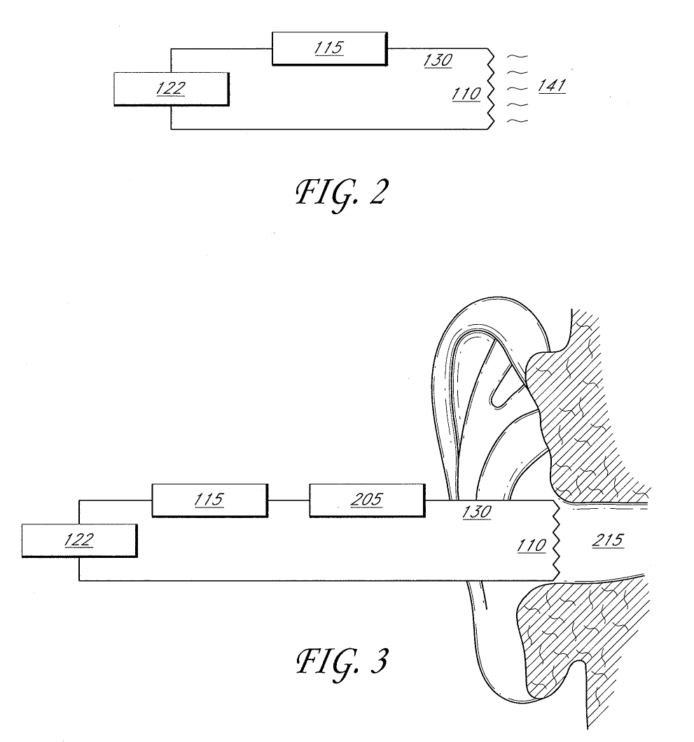

Numerous types of devices may be used to generate heat directly to the vestibular apparatus of the inner ear of a patient to elicit a physiological response, including electrical stimulators, lasers and infrared devices. Some embodiments use one or more heating elements, such as resistive heating coils. FIG. 2 illustrates one embodiment of a circuit for supplying heat to the vestibular apparatus, and includes a thermal-transferring element, such as a resistor 110, a power supply 122, and a switch 115. The different electrical elements are connected by one or more conductive wires 130.

In one embodiment, resistor 110 is placed proximate to the ear canal of a patient to gently warm the ear, thereby generating a thermal differential between the ear canal and the vestibular apparatus of the inner ear. Not to be limited by theory, with such a thermal differential, thermal energy will be transferred across the middle ear, causing thermal stimulation in the vestibular apparatus and thereby suppressing the patient's appetite.

Resistor 110 may include a heating coil comprised of any resistive material or combination of resistive materials, such as a coiled nickel and chromium Nichrome™ wire insulated with an appropriate material. The resistor 110 may be a heating coil that is, in some embodiments, between 1 and 3 inches in length, such as between 1.5 and 2 inches in length. By turning on power supply 122, resistor 110 may generate heat which may be transferred to the inner ear. The resistor 110 may be placed proximate to a portion of the ear canal when the invention is in use. In some embodiments, the resistor 110 may touch a portion of the ear canal, while in other embodiments, the resistor 110 will not touch any portion of the ear canal. It is possible for the resistor 110 to be outside of the ear completely so long as enough heat is applied to create a thermal differential to stimulate the vestibular apparatus.

The resistor 110 will generate heat at or near the ear canal, and thermal energy 141 will be transmitted to the inner ear, thereby inducing caloric stimulation of the vestibular apparatus. In some embodiments, the resistor 110 is configured to heat the inner ear to no more than about 10, 9, 8, 7, 6, 5, 4, 3, 2, 1, or less degrees Celsius above normal body temperature (e.g., 37 degrees Celsius).

In some embodiments, the vestibular stimulation may be provided for a specified period of time, such as no more than about 24 hours, 12 hours, 6 hours, 4 hours, 3 hours, 2 hours, 1 hour, 45 minutes, 30 minutes, 20 minutes, 15 minutes, 10 minutes, 8 minutes, 7 minutes, 5 minutes, 4 minutes, 3 minutes, 2 minutes, 1 minute, 45 seconds, 30 seconds, 20 seconds, 15 seconds, 10 seconds, 5 seconds, 3 seconds, 2 seconds, 1 second, or less. In some embodiments, the vestibular stimulation may be provided for at least about 1, 2, 3, 4, or 5 seconds, or more.

In some embodiments, the stimulus may be of the same intensity and/or duration during one or more stimulus cycles. In other embodiments, the stimulus may be of varying intensity and/or duration during one or more stimulus cycles. As used herein, the term “stimulus cycle” refers to a period of stimulation (e.g., by heating the inner ear with a thermal stimulus such as heat from a resistor or other heating element) followed by a period of non-stimulation (e.g., by removing the thermal stimulus). In some embodiments, the duration of the period of stimulation is not the same as the duration of the period of non-stimulation in a single cycle. Because constant vestibular stimulation can lead to desensitization and loss of response, in some embodiments, the thermal stimulus can be applied intermittently. In some embodiments of the invention, a thermal stimulus of no more than about 360, 240, 180, 120, 90, 75, 60, 45, 30 seconds or less can be effectively applied every five, ten, fifteen, twenty, thirty, or more minutes to a patient. The thermal stimulus may be applied, for example, for no more than about 100, 80, 60, 50, 40, 30, 25, 20, 15, 12, 10, 8, 5, 4, 3, 2, or 1 stimulus cycles per day. In one embodiment, a thermal stimulus that is between 15 and 60 seconds may be applied intermittently at periods between, for example 3 and 10 minutes. In some embodiments, the stimulus may be provided no more than about 2 hours, 1 hour, 45 minutes, 30 minutes, 20 minutes, 15 minutes, 10 minutes, 5 minutes, or less prior to one or more meals, or prior to every meal. In some embodiments, the stimulus may be provided more frequently and/or of a greater duration near normal mealtimes and less frequently and/or of a lesser duration in between normal mealtimes, such as, for example, any of the frequencies noted above. In some embodiments, the system is configured to stimulate the vestibular system such that the patient's daily caloric intake while being treated is at least about 5%, 10%, 15%, 30%, 35%, 30%, 35%, 40%, 50% less than the patient's caloric intake prior to treatment.

The rate at which the resistor 110 will heat depends on the material used in the resistor and the length of the coils used in the resistor, as well as the length of the conductive wire 130. In general, the longer the length of the conductive wire 130, the slower the heating time to the desired temperature. In some embodiments, the resistor 110 may be heated to the desired stimulus temperature of greater than 40 degrees Celsius in less than 30 seconds, depending on the material that is used for the resistor.

As the sensitivity of each individual patient's vestibular system to a particular thermal stimulus may vary, it will be appreciated that the temperature settings, duration of which the thermal stimulus is applied, duration in between thermal stimuli, and number of cycles per day may be varied by the physician according to the desired clinical result.

Power supply 122 supplies a voltage and generates heat in the resistor 110. Power supply 122 may be a battery, in the range of, for example, 1.5 to 9 volts, although higher voltage supplies may be used. In a preferred embodiment, the power supply 122 is a 1.5 V alkaline power cell battery. In another embodiment, the power supply 122 may include one or more miniaturized (e.g., “button cell”) batteries to supply a voltage. Other power sources, such as, for example, solar, mechanical, or the patient's own thermal energy may be employed. Located in between power source 122 and resistor 110 is switch 115. This switch 115 acts as an on/off switch for the device and may deactivate the thermal energy generated in the resistor 110.

FIG. 3 illustrates a second embodiment of a circuit of the present invention, the invention being inserted proximate to the ear canal 215 of a patient. According to FIG. 2, the circuit includes a control unit 205, which can be located in between switch 115 and resistor 110, and kept outside the ear in some embodiments. In some embodiments, the control unit 205 may be used to control the output temperature of heat in the resistor 110, via, for example, a CPU so as to induce a sense of anorexia in a patient without undesired side effects such as nausea, vomiting, vertigo, or nystagmus. For example, in some embodiments, the control unit 205 is set to heat the ear canal 215 of a patient to a particular temperature, such as between about 37° C. and 40° C. The control unit 205 may also be used to ensure that appropriate heat is released at specific time intervals or incremental levels such that the heat released will not be excessive as to induce unwanted effects as mentioned. The control unit 205, in some embodiments, may be programmed to maintain the resistor at a specific temperature. The control unit 205 may also be programmed to control the switch 115 to be on and off in various cycles, such that the amount of heat generated will fluctuate during the treatment process. In addition, the control unit 205 may include a dial or other adjustable control to adjust the temperature which may be operated by either a patient or physician. In some embodiments, the control unit communicates wirelessly with a sensor in the resistor to control temperature sufficient to induce anorexia without other undesired side effects as noted above. The control unit 205 may also be attached to an input device, such as a feedback sensor, the sensor being used to detect physiological conditions of a patient and transmit signals to the control unit to ensure appropriate heat in the resistor 110. The control unit or sensor device may include an audible or visual component designed to alert the patient or doctor when the resistor has reached a certain temperature. The device may also be programmed to shut off as a safety feature if a certain temperature is exceeded. In some embodiments, the control unit 205 functions without a sensor, and provides for a predetermined pattern of thermal stimulation for a set amount of time to the patient. In other embodiments, rather than using control unit 205 to moderate the resistor's temperature, the resistor's temperature may be regulated by altering the length of the heating coil wire, altering the composition of the conductive wire 130 used in the circuit, altering the voltage of the battery 122 or by using a potentiometer in the circuit. In some embodiments, the device including controls may be included or integrated as part of a system with other functionality, such as, for example, a computer, audio player such as an MP3 player, with hardware and/or software temperature cycle controls as noted above.

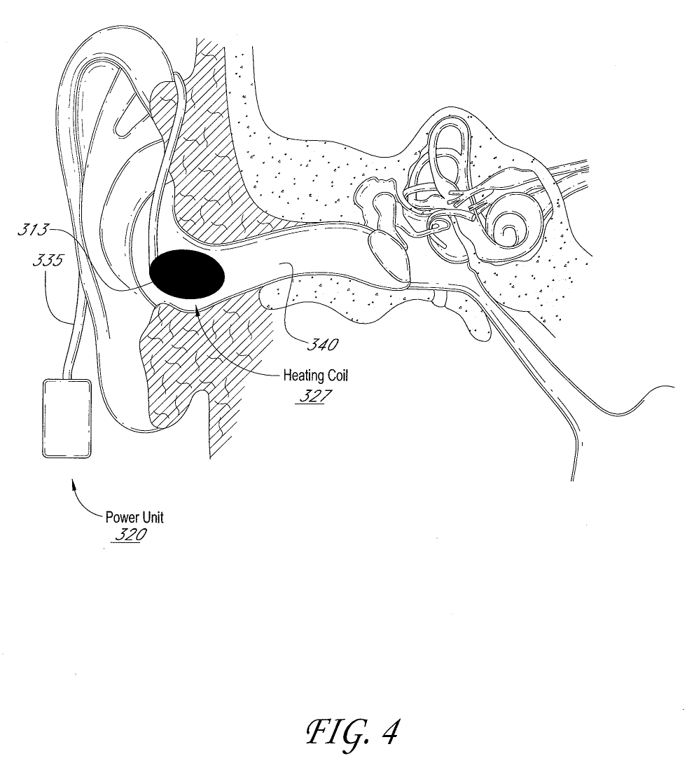

FIG. 4 illustrates one embodiment of the present invention as shown in the ear of a patient. As shown in FIG. 4, the invention includes a power unit 320 (e.g. a battery) and a heating coil 327 embedded in an ear interface, such as an ear plug 313. Ear plug 313 may act as an insulator and cover for heating coil 327 and may add comfort to the patient while the invention is in use. It may also serve to transmit the heat from the resistor to the patient's ear. The ear plug 313 may be composed of a soft rubber material or other material that does not significantly impede the thermal energy from being emitted in the ear. Such materials may include but are not limited to foam, silicone, nylon, latex or various combinations of these. Ear plug 313 may also be of varying shapes and sizes, depending on the shape and size of the patient's ear. In one embodiment, ear plug 313 is composed of an occlusive material, and allows the ear plug to act as a barrier to thermal outflow for heat generated within the ear. In another embodiment, ear plug 313 may be non-occlusive and specifically designed to shape an individual ear.

As shown in FIG. 4, in a preferred embodiment, the power unit 320 is provided outside the ear of the patient, which allows for easy recharging or replacing of the power supply, as well as for maximum comfort for the patient. The power unit 320 may also be attached to a sensor device and/or control unit, both of which may also be found outside the ear of the patient to maximize comfort for the patient. In some embodiments, the power unit, sensor or control may all be placed in or near the ear canal of the patient.

When in use, the invention should preferably only be minimally obtrusive to the patient, as it need not be placed into the inner ear or deep inside the ear canal of a patient in order to stimulate the vestibular apparatus. As shown in FIG. 4, in one embodiment, the heating coil 327 need only be placed in an ear canal 327, close to the external opening of a patient's ear. In some embodiments, heating coil 327 need not be placed in the ear at all and will be completely non-invasive, so long as enough heat is able to reach the inner ear of the patient. Preferably, the apparatus is lightweight and portable, and may be attached to at least one of the patient's ears by use of a clip 335 as shown in FIG. 3. In other embodiments, it may be attached to an ear as with an over-the-ear or in-ear hearing aid. For example, the apparatus may be worn behind-the-ear (BTE) such that it is attached to an ear mold that fits mostly in the concha; as an in-the-ear (ITC) device which largely fits in the auricle and concha areas; as an in-the-canal (ITC) device which fits largely in the concha area and extends into the ear canal; or as a completely-in-the-canal (CIC) device which fits completely within the ear canal past the opening of the ear, or at any position described in U.S. Pat. No. 7,310,426 to Shennib et al., which is hereby incorporated by reference in its entirety. In some embodiments, the system may include a heating patch that can be attached proximate to the mastoid bone. One of skill in the art will appreciate that other ways of attachment may be devised such that a patient may comfortably wear the device.

In one embodiment, the invention will provide caloric stimulation to the vestibular apparatus either unilaterally or bilaterally. More specifically, in one embodiment, the invention will generate a thermal differential to stimulate the vestibular apparatus in one or more inner ears by placing a heating coil in the ear canal to thermally stimulate locations at or near the vestibular apparatus

When in use, the temperature of the heating coil 327 may be between, for example, 35 to 60 degrees Celsius, such as 35, 40, 45, 50, 55, or 60 degrees Celsius with a temperature of less than about 40 degrees Celsius preferred in some embodiments to prevent burning, discomfort, etc. The amount of time required to generate a state of anorexia in some embodiments will vary depending on the temperature of the heating coil 327, with the higher the temperature of the heating coil, the more quickly a state of anorexia is induced.

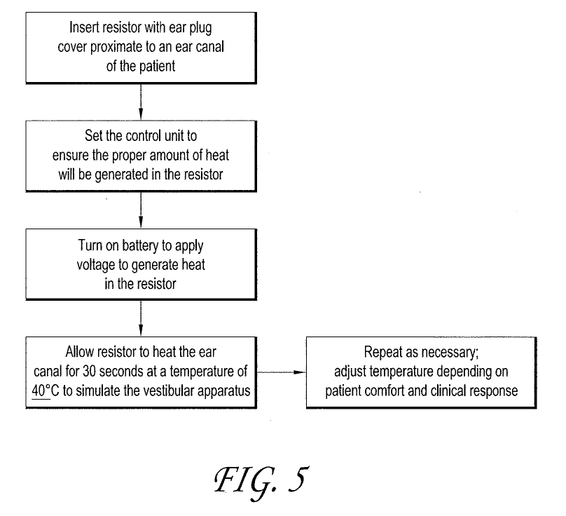

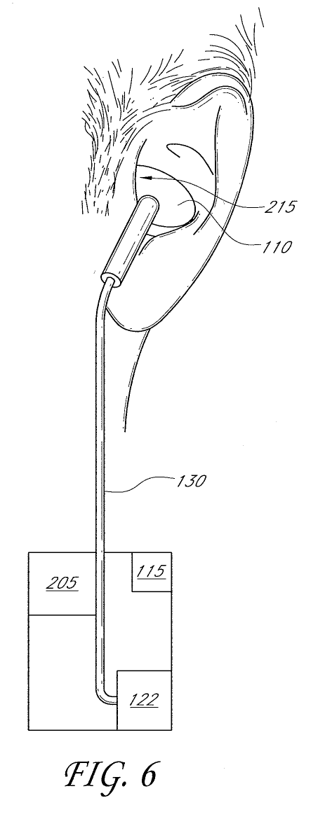

FIG. 5 shows a schematic diagram of one embodiment of a method used by a physician to treat a patient. First, the physician will insert a resistor, having a coil length of approximately 2 inches, into the ear of a patient such that the resistor is proximate to an ear canal of the patient. Second, the physician will program a control unit so as to ensure that sufficient heat is being generated in the resistor to induce anorexia and to ensure that the resistor does not cause pain, discomfort, or overstimulation of the vestibular system. The control unit should be set to produce the desired clinical results. Once the control unit is set, the physician may then activate the system to supply a voltage and generate heat in the resistor, which is connected to the battery by a conductive wire. Using a 1.5 V battery and a short conductive wire of approximately 2 inches, the resistor can be allowed to heat the inner ear for approximately 30 seconds at a temperature of approximately 40° C. or less to induce a state of anorexia. Any other appropriate length of conductive wire may be used depending on the desired clinical result, such as no more than about 12″, 9″, 6″, 5″, 4″, 3″, 2.5″, 2″, 1.5″, 1″ (inch) or less. The stimulus can be applied for any number of times depending on the desired clinical result.

### EXAMPLE

An 8-week study was conducted on five obese subjects (BMI>30) using an auricular thermoregulation device. Subjects included three females and two males. The average weight at study entry was 279 pounds (BMI=43.7). All subjects were offered dietary weight loss counseling, but no specific diet was prescribed. The study consisted of a 4-week control period, in which subjects returned for weekly weight measurements but did not use the auricular thermoregulation device. After 4 weeks, subjects were given an auricular thermoregulation device comprising a control switch and heating coil for stimulating the vestibular apparatus. The subjects were advised to insert the heating coil in a single ear canal and to activate it as needed during periods of hunger by maintaining constant pressure on the control switch until the heating coil was detectably warm. The heating coil used reached a temperature of approximately 45 to 50 degrees Celsius in about 15 seconds. Subjects were encouraged to initiate the heating activity for no more than about 30 seconds with intervals or no more than approximately every 5 or 10 minutes as a means of reducing potential tolerance.

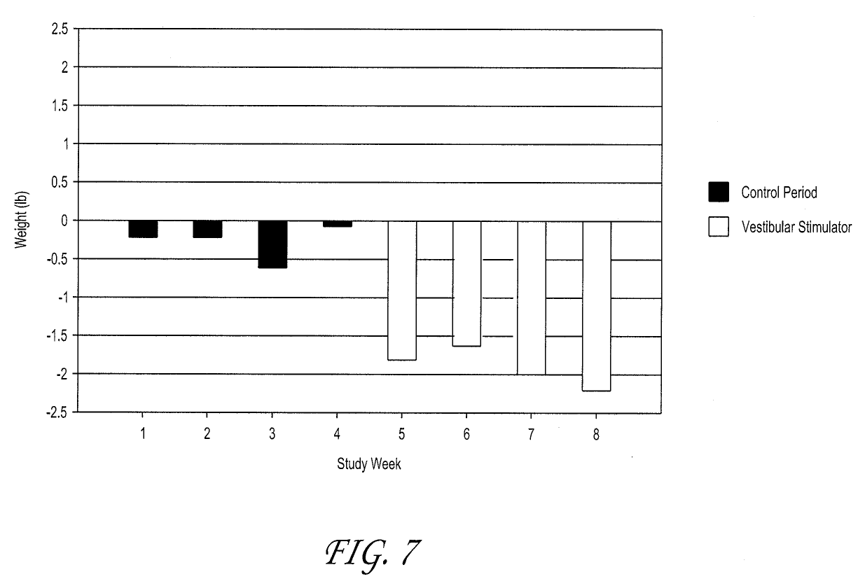

Four of the five subjects felt the device helped to reduce their appetite, while one of the subjects complained that the device caused mild nausea. FIG. 7 shows a bar graph illustrating the average weekly weight-loss of the five subjects during the eight-week study. The bars indicate the average weight v. the baseline weight for all subjects. During the control period (the first 4 weeks), the subjects' weekly weight loss average from the baseline weight was approximately 0.2, 0.2, 0.6 and 0 lbs. During the 4 weeks of device intervention, the subjects' weekly weight loss average from the baseline weight was approximately 1.8, 1.6, 2.0 and 2.2 lbs during each of weeks 5, 6, 7 and 8, respectively.

In summary, after 4 control weeks, the subjects had lost no measurable weight. However, during the 4 weeks of device intervention, the subjects had a 4-week average weight loss of approximately 2.2 lbs (p=0.05), thereby demonstrating a statistically significant average weight loss for a relatively short period of device use.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Additionally, the skilled artisan will recognize that any of the above-described methods can be carried out using any appropriate apparatus. Further, the disclosure herein of any particular feature in connection with an embodiment can be used in all other disclosed embodiments set forth herein. Thus, it is intended that the scope of the present invention herein disclosed should not be limited by the particular disclosed embodiments described above.

What is claimed is:

1. A method of suppressing appetite of a person desiring weight management, comprising:
identifying a person desiring weight management;
placing a heating device proximate to an inner ear of the person, the heating device being electrically connected to a power supply;
turning on the power supply to generate heat in the heating device;
applying the heat from the heating device for a predetermined period of time to heat the inner ear; and
controlling the heat from the heating device using a control unit such that the person's appetite is suppressed.

2. The method of claim 1, wherein the heating device is placed proximate to the inner ear canal of the person.

3. The method of claim 1, wherein the heating device is maintained at a temperature of greater than about 37° C. but less than about 40° C.

4. The method of claim 1, wherein the inner ear is heated to a temperature of greater than about 37° C. but less than about 40° C. for no more than about 15 seconds.

5. The method of claim 1, wherein the inner ear is heated to a temperature of greater than about 37° C. but less than about 40° C. for no more than about 30 seconds.

6. The method of claim 1, wherein the inner ear is heated to a temperature of greater than about 37° C. but less than about 40° C. for no more than about 60 seconds.

7. The method of claim 1, further comprising the step of stopping the heating of the inner ear after the predetermined period of time for heating.

8. The method of claim 7, further comprising repeating the step of applying heat from the heating device to the inner ear after stopping the heating of the inner ear.

9. The method of claim 8, wherein stopping the heating of the inner ear is for no more than about 15 minutes before repeating the step of applying heat from the heating device to the inner ear.

10. The method of claim 8, wherein stopping the heating of the inner ear is for no more than about 5 minutes before repeating the step of applying heat from the heating device to the inner ear.

11. The method of claim 7, wherein the steps of heating and stopping the heating of the inner ear comprise a stimulation cycle.

12. The method of claim 11, wherein the stimulation cycle is repeated at least 10 times in a day.

13. The method of claim 1, wherein the suppression of the person's appetite is transient.

14. A method for inducing anorexia in a patient, comprising:
inserting a resistor proximate to an ear canal of the patient, the resistor being electrically connected to a power supply;
turning on the power supply to generate heat in the resistor;
applying the heat from the resistor to heat the ear canal; and
controlling the heat from the resistor using a control unit such that a sensation of anorexia is induced in a patient.

15. The method of claim 14, wherein applying heat from the resistor heats the ear canal to a temperature of greater than about 37° C. but less than about 40° C. for between about 3 seconds and 30 seconds.

16. A method of heating the inner ear of a person, comprising:
placing a resistor proximate to an ear canal of the person, the resistor being electrically connected to a power supply;
turning on the power supply to generate heat in the resistor;
applying the heat from the resistor to heat the ear canal; and
controlling the heat from the resistor using a control unit.

17. The method of claim 16, wherein heating the inner ear causes appetite suppression in the person.

18. The method of claim 17, wherein the appetite suppression is transient.

19. The method of claim 18, wherein the transient appetite suppression lasts no more than about 5 minutes.

20. The method of claim 18, wherein the transient appetite suppression lasts no more than about 1 hour.

21. The method of claim 16, wherein the resistor is heated to a temperature between about 45° C. and 50° C.

22. An apparatus for managing weight in a person, comprising:
a heating element configured to be placed proximate to an ear of the person; and
a control unit configured to control the amount and duration of heat being generated in the heating element so as to induce a sensation of anorexia in a patient when the heating element is placed proximate to the ear of the person.

23. The apparatus of claim 22, wherein the heating element comprises a heating coil.

24. The apparatus of claim 23, wherein the heating coil is a resistance wire between 1.5-2.5 inches in length.

25. The apparatus of claim 23, wherein the heating coil is sized to fit into an ear canal of a person.

26. The apparatus of claim 23, wherein the battery has a voltage of between about 1.5 volts and 9 volts.

27. The apparatus of claim 23, wherein the heating coil is covered by an insulative material.
